(19) United States
(12) Patent Application Publication
(10) Pub. No.: US 2021/0010048 A1
(43) Pub. Date: Jan. 14, 2021

(54) METHOD OF PREPARING CORN GLYCOPEPTIDES, AND PRODUCT AND USE THEREOF

(71) Applicant: QIQIHAR UNIVERSITY, Qiqihar (CN)

(72) Inventors: Xiaolan LIU, Qiqihar (CN); Xiaojie WANG, Qiqihar (CN); Xiqun ZHENG, Qiqihar (CN)

(73) Assignee: QIQIHAR UNIVERSITY, Qiqihar (CN)

(21) Appl. No.: 16/922,377

(22) Filed: Jul. 7, 2020

(30) Foreign Application Priority Data
Jul. 9, 2019 (CN) 201910613648.9

(51) Int. Cl. C12P 21/06; C12P 21/00; A23J 3/34; C07K 1/34; C07K 1/36

(52) U.S. Cl. CPC C12P 21/06; C12P 21/005; C07K 1/36; C07K 1/34; A23J 3/346

(57) ABSTRACT

A method for preparing corn glycopeptides, and a product and use thereof, and belongs to the technical field of food and medicine production. The method for preparing corn glycopeptides includes the preparation of corn peptides powder and the glycosylation reaction between corn peptides and amino sugar; the preparation raw material of the corn peptides powder is a corn protein rich in branched chain amino acids; and the corn peptides and the amino sugar are subjected to the glycosylation reaction catalyzed by a transglutaminase, and finally the corn glycopeptides is obtained. The method for preparing corn glycopeptides provided by the present disclosure is simple; and the prepared corn glycopeptides have antagonistic and protective effects on alcoholic liver injury, and can be applied to the preparation of related foods, medicines and health care products.

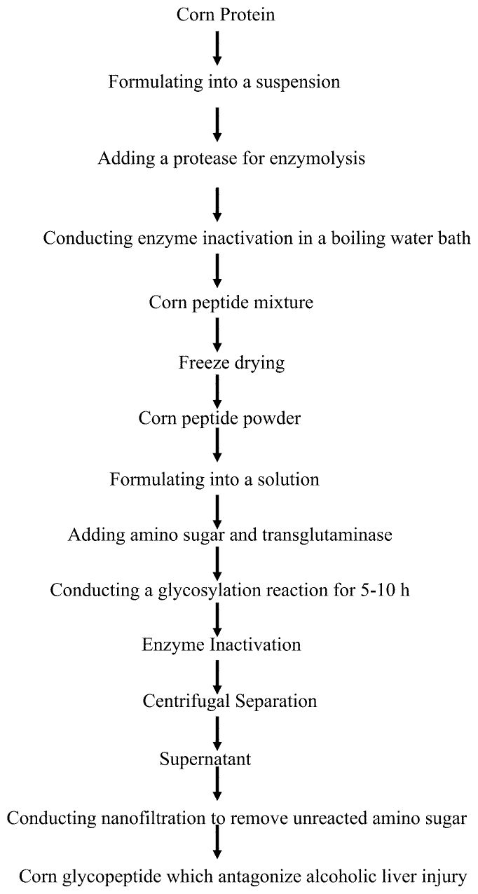

### BACKGROUND

### 1. Field of the Disclosure

The present disclosure belongs to the technical field of food and medicine production, and particularly relates to a method for preparing corn glycopeptides, and a product and use thereof.

### 2. Discussion of the Background Art

Many people drink alcohol on various social occasions for various reasons. Toasting, drink-urging or drinking as being afraid of hurting sb.'s feelings often lead to excessive drinking of human. Long-term drinking can lead to alcohol addiction of human, and thus alcoholism. Long-term alcoholism is more harmful to liver. After alcohol intake, the alcohol is rapidly absorbed through the gastrointestinal tract into blood, most of the alcohol is metabolized in the liver, and only 2%-10% of the alcohol is excreted by the kidney and the lung. Alcohol has an obvious toxic effect on liver cells. Alcoholic fatty liver, alcoholic hepatitis, and alcoholic liver cirrhosis, and even canceration can be caused by long-term alcoholism.

Clinically, western medicine treatment drugs for alcoholic liver injury usually increase the liver burden. Traditional Chinese medicine has the characteristics of relatively small side effects, safety and effectiveness, and can be more acceptable by the public. However, although there are several kinds of traditional Chinese medicine preparations for treating alcoholic liver injury clinically at present, there are fewer raw material drugs with the homology of medicine and food in the components, these raw material drugs still have side effects when taken frequently, are slow to take effect, and have a low curative effect and unsatisfactory results.

### SUMMARY

In view of the problems existing in the background art, an objective of the present disclosure is to provide corn glycopeptides capable of effectively antagonizing alcoholic liver injury, and a preparation method and use thereof.

The present disclosure provides a method for preparing corn glycopeptides, including the following steps:

(1) hydrolyzing a corn protein suspension, conducting liquid-solid separation, and drying a liquid phase to obtain corn peptides powder;

(2) mixing the corn peptides powder with water to formulate a corn peptides solution with a mass-volume concentration of 2-15%;

(3) adding amino sugar into the corn peptides solution to obtain a reaction base solution; wherein the addition amount of the amino sugar is 2-5 times of the mass of the corn peptides powder;

(4) adding a transglutaminase into the reaction base solution to enable a glycosylation reaction between the corn peptides and the amino sugar, so as to obtain the corn glycopeptides after the reaction is finished; wherein the addition amount of the transglutaminase is 40-80 U of the transglutaminase per 1 g of the corn peptides powder.

Preferably, the hydrolytic enzyme in the step (1) includes one or more of an Alcalase protease, a Protamex protease, and a Flavourzyme protease.

Preferably, when the hydrolytic enzyme is the Alcalase protease, the hydrolysis temperature is 55-65° C., the pH value of the hydrolysis is maintained at 8.2-8.8, and the hydrolysis time is 1-3 h.

Further preferably, the maintaining of the pH value is achieved by adding 0.4-0.6 mol/L of the NaOH solution dropwise.

Preferably, after the hydrolysis in the step (1), the method further includes primary enzyme inactivation, wherein the temperature of the primary enzyme inactivation is 90-105° C., and the time of the primary enzyme inactivation is 10-20 min.

Preferably, the manner of solid-liquid separation in the step (1) is centrifugation, the rotation speed of the centrifugation is 3000-5000 r/min, and the centrifugation time is 10-20 min.

Preferably, after the glycosylation reaction in the step (4) is finished, the method further includes the steps of centrifuging the reaction solution, taking the supernatant after the centrifuging, and performing nanofiltration of the supernatant; wherein the nanofiltration can cut off molecules with molecular weights ≤300 Da.

Preferably, for the nanofiltration, the pressure is 15-25 Bar, and the temperature is 18-22° C., and the solution volume after the nanofiltration is ⅓-⅔ of the solution volume before the nanofiltration.

Preferably, the temperature of the glycosylation reaction in the step (4) is 42-46° C., the pH value of the glycosylation reaction is maintained at 7.8-8.2, and the time of the glycosylation reaction is 5-10 h.

Further preferably, the maintaining of the pH value is achieved by adding 3-5 mol/L of the NaOH solution dropwise.

Preferably, the glycosylation reaction further includes secondary enzyme inactivation, the temperature of the secondary enzyme inactivation is 80-90° C., and the time of the secondary enzyme inactivation is 3-8 min.

The present disclosure provides a corn glycopeptides-containing product prepared by the aforementioned preparation method, wherein the mass concentration of corn protein in the product is 70%-80%, and the protein includes corn glycopeptides and corn peptides; and the amount of glucosamine conjugated to corn peptides is 100-150 mg per 1 g of the corn peptides.

The present disclosure further provides application of the aforementioned corn glycopeptides or corn glycopeptides-containing product in the preparation of health-care food, drugs or health care products for antagonizing alcoholic liver injury.

Beneficial effects: the present disclosure provides a method for preparing corn glycopeptides, including the following steps: (1) hydrolyzing a corn protein suspension, conducting liquid-solid separation, and drying a liquid phase to obtain corn peptides powder; (2) mixing the corn peptides powder with water to formulate a corn peptides solution with a mass-volume concentration of 2-15%; (3) adding amino sugar into the corn peptides solution to obtain a reaction base solution; wherein the addition amount of the amino sugar is 2-5 times of the mass of the corn peptides powder; and (4) adding a transglutaminase into the reaction base solution to enable a glycosylation reaction between the corn peptides and the amino sugar, so as to obtain a final reaction solution containing the corn glycopeptides after the reaction is finished; wherein the addition amount of the transglutaminase is 40-80 U of the transglutaminase per 1 g of the corn peptides powder. The method for preparing corn glycopeptides provided by the present disclosure is simple; and the prepared corn glycopeptides have antagonistic and protective effects on alcoholic liver injury, can be applied to the preparation of related foods, medicines and health care products, and has no toxic and side effects.

### BRIEF DESCRIPTION OF THE DRAWING

A FIGURE of a process flow chart of producing corn glycopeptides according to Example 1 of the present disclosure.

### DETAILED DESCRIPTION OF THE PREFERRED EMBODIMENT

The present disclosure provides a method for preparing corn glycopeptides, including the following steps:

(1) hydrolyzing a corn protein suspension, conducting liquid-solid separation, and drying a liquid phase to obtain corn peptides powder;

(2) mixing the corn peptides powder with water to formulate a corn peptides solution with a mass-volume concentration of 2-15%;

(3) adding amino sugar into the corn peptides solution to obtain a reaction base solution; wherein the addition amount of the amino sugar is 2-5 times of the mass of the corn peptides powder;

(4) adding a transglutaminase into the reaction base solution to enable a glycosylation reaction between the corn peptides and the amino sugar, so as to obtain the corn glycopeptides after the reaction is completed; wherein the addition amount of the transglutaminase is 40-80 U of the transglutaminase per 1 g of the corn peptides powder.

In the present disclosure, the corn peptides powder is prepared by taking the corn protein as a raw material. The corn protein is rich in branched chain amino acids, and the corn peptides powder prepared by taking the corn protein as the raw material has a potential liver protection activity. In the present disclosure, the corn protein is preferably zein. The present disclosure has no specific limitation on the source of the corn protein, and a conventional commercially-available product in the art can be used. In an embodiment of the present disclosure, the corn protein is purchased from Sigma.

In the present disclosure, the corn protein is hydrolyzed first. In the present disclosure, the hydrolysis operation includes first formulating an aqueous suspension of the corn protein, wherein the mass volume concentration of the corn protein in the aqueous suspension is preferably 5-15%, and more preferably 10%. In the present disclosure, the hydrolysis enzyme preferably uses one or more of an Alcalase protease, a Protamex protease and a Flavourzyme protease, and more preferably uses the Alcalase protease. In the present disclosure, before the Alcalase protease is added, preferably the aqueous suspension of the corn protein is heated to the hydrolysis temperature, and the pH value of the aqueous suspension of the corn protein is adjusted to the hydrolysis pH value. In the present disclosure, the hydrolysis temperature is preferably 55-65° C., and more preferably 60° C. The pH value of the hydrolysis is preferably 8.2-8.8, and more preferably 8.5. In the present disclosure, the hydrolysis pH value is preferably realized by adding the NaOH solution dropwise, and the concentration of the NaOH solution is preferably 0.4-0.6 mol/L, and more preferably 0.5 mol/L. In the present disclosure, the added mass concentration of the Alcalase protease is preferably 1%-5%, and more preferably 3%. The present disclosure has no specific limitation on the source of the Alcalase protease, and a conventional commercially-available product in the field can be used. In an embodiment of the present disclosure, the Alcalase protease is purchased from Novozymes, Denmark. In the present disclosure, the hydrolysis time is preferably 1-3 h, and more preferably 2 h.

In the present disclosure, after the hydrolysis is completed, preferably the hydrolysate is subjected to the enzyme inactivation operation. In the present disclosure, the temperature of the enzyme inactivation is preferably 90-105° C., and more preferably 100° C. The enzyme inactivation time is preferably 10-20 min, and more preferably 15 min.

After the hydrolysis, the corn protein is fully hydrolyzed into the corn peptides and dissolved in the hydrolysate. In the present disclosure, the hydrolysate is subjected to a solid-liquid separation treatment, and the filtrate is dried to obtain the corn peptides powder. In the present disclosure, the solid-liquid separation manner is preferably centrifugation, and the drying manner is preferably freeze drying. In the present disclosure, the rotational speed of the centrifugation is preferably 3000-5000 r/min, and more preferably 4000 r/min. The centrifugation time is preferably 10-20 min, and more preferably 15 min. The freeze-drying temperature is preferably −70 to −90° C., and more preferably −80° C. The freeze-drying time is preferably 36-60 h, and more preferably 48 h. The corn peptides powder prepared by the method provided by the present disclosure has a high ratio of the branched chain amino acids to the aromatic amino acids, molecular weights concentrated in the range of 500-1000 Da, and the characteristics of liver protection and easy digestion and absorption.

In the present disclosure, after the corn peptides powder is obtained, the corn peptides powder is mixed with water to obtain the corn peptides solution. In the present disclosure, the mass volume concentration of the corn peptides solution is 2-15%, preferably 3-10%, and more preferably 3.5-5%. In the present disclosure, after the corn peptides solution is formulated, the amino sugar is added into the corn peptides solution to obtain a glycosylation reaction base solution. In the present disclosure, the amino sugar is preferably D-glucosamine hydrochloride. The addition amount of the amino sugar is preferably 2-5 times the mass of the corn peptides powder, more preferably 2.5-3.5 times the mass of the corn peptides powder, and more preferably 3 times the mass of the corn peptides powder. In the present disclosure, after the glycosylation reaction base solution is obtained, the transglutaminase is added into the glycosylation reaction base solution to enable the glycosylation reaction between the corn peptides and the amino sugar. In the present disclosure, the mass ratio of the added amount of the transglutaminase to the corn peptide powder is 40-80 U: 1 g, preferably 50-60 U: 1 g, and more preferably 55 U: 1 g. The temperature of the glycosylation reaction is preferably 42-46° C., and more preferably 44° C. The pH value of the glycosylation reaction is preferably 7.8-8.2, and more preferably 8.0. In the present disclosure, the pH value of the glycosylation reaction is preferably realized by adding the NaOH solution dropwise, and the concentration of the NaOH solution is preferably 3-5 mol/L, and more preferably 4 mol/L. The present disclosure has no specific limitation on the sources of the amino sugar and the transglutaminase, and a conventional commercially-available products in the art can be used. In an embodiment of the present disclosure, the amino sugar is purchased from Sangon Biotech (Shanghai) Co., Ltd, and the transglutaminase is purchased from Yiming biological technology Limited, Taixing.

In the present disclosure, after the glycosylation reaction is completed, preferably the final reaction solution containing the corn glycopeptides is subjected to the enzyme inactivation operation, and in the present disclosure, the enzyme inactivation temperature is preferably 80-90° C., and more preferably 85° C. The enzyme inactivation time is preferably 3-8 min, and more preferably 5 min.

In the present disclosure, after the glycosylation reaction is completed, it preferably includes the steps of centrifuging the reaction solution, taking the supernatant after the centrifuging, and performing nanofiltration of the supernatant.

In the present disclosure, the rotational speed of the centrifugation is preferably 3000-5000 r/min, and more preferably 4000 r/min. The centrifugation time is preferably 10-20 min, and more preferably 15 min. The supernatant is obtained after the centrifuging, and then the supernatant is subjected to nanofiltration. In the present disclosure, the nanofiltration is preferably conducted by employing a nanofiltration membrane, and the nanofiltration membrane can cut off molecules with molecular weights ≤300 Da. In the present disclosure, the nanofiltration pressure is preferably 15-25 Bar, and more preferably 20 Bar; and the nanofiltration temperature is preferably 18-22° C., and more preferably 20° C. The solution volume after the nanofiltration is reduced, and the solution volume is preferably ⅓-⅔ of the solution volume before the nanofiltration, and more preferably ½ of the solution volume before the nanofiltration. Compared with the corn glycopeptides before the nanofiltration, the corn glycopeptides after the nanofiltration has a conductivity reduced by more than 15% and a sugar content reduced by more than 7%, thereby achieving the purpose of desalting and removing the unreacted amino sugar.

The present disclosure provides a corn glycopeptides-containing product prepared by the aforementioned preparation method. In the present disclosure, the mass concentration of the protein in the product is 70%-80%. The protein includes corn glycopeptides and corn peptides; and the amount of glucosamine conjugated to corn peptides is 100-150 mg per 1 g of the corn peptides.

The present disclosure further provides application of the corn glycopeptides or corn glycopeptides-containing product prepared by the aforementioned preparation method in the preparation of health-care food, drugs or health care products for antagonizing alcoholic liver injury. Experiments indicate that, the corn glycopeptides provided by the present disclosure has obvious antagonism and protection effects on alcoholic liver injury; and compared with an alcohol model group, the corn glycopeptides obviously reduced the hepatic index and the activities of ALT and AST in the serum and the content of ROS in the liver of rats, while obviously increased the activities of SOD and CAT in the liver.

The method for preparing corn glycopeptides, and a product and application as provided by the present disclosure will be described in detail below with reference to embodiments, but they cannot be understood as limiting the claimed scope of the present disclosure.

### Example 1

Preparation of Corn Glycopeptides;

1. Preparation of corn peptides: hydrolysis of corn protein catalyzed by the Alcalase protease: the zein of 10 g was taken and placed in a 150 mL beaker, added with 100 mL of distilled water, and stirred evenly to formulate a suspension with a substrate concentration of 10% (w/v). After a rotor of an appropriate size was added into the beaker, the beaker was placed at the center of a magnetic stirrer in a 60° C. water bath, and the electrode of a pH meter was inserted into the suspension. 0.5 mol/L NaOH was added at a small amount for several times with a pipette gun to adjust the pH of suspension to 8.5 until the pH value was maintained for 3 min, and then added with 0.3 g of the Alcalase protease to start an enzymolysis reaction. During the enzymolysis process, the pH was maintained at 8.5 by adding 0.5 mol/L of NaOH continuously, and after enzymolysis for 2 h, the suspension was taken out and immediately put into a boiling water bath to conduct enzyme inactivation for 15 min, cooled to room temperature, and then centrifuged at 4000 r/min for 15 min. The supernatant was taken, subpackaged into small boxes, frozen in a −80° C. refrigerator, and freeze-dried for 48 h to obtain the corn peptides powder.

2. Glycosylation modification of corn peptides by glucosamine hydrochloride

(1) The prepared corn peptides powder was formulated into a corn peptides solution with a substrate concentration of 3.5%, added with D-glucosamine hydrochloride according to a corn peptide: D-glucosamine hydrochloride ratio of 1:3 (w/w), and was adjusted with 2 mol/L NaOH until the pH value was 8.0.

(2) The solution was added with the transglutaminase according to the added enzyme amount of 55 U/g of protein, sealed with a film, and put into a constant temperature water bath oscillator at 44° C., and the glycosylation modification reaction was started. The solution was taken out every 1 h and adjusted with 4 mol/L NaOH until the pH was 8.0.

(3) After the glycosylation reaction 7 h, immediately the solution was subjected to enzyme inactivation in an 85° C. water bath for 5 min.

(4) The solution was cooled to room temperature, and subjected to desalting and removing D-glucosamine treatment with a nanofiltration membrane with a cut-off molecular weight of 300 Da to obtain the corn glycopeptides, wherein the nanofiltration parameters were a solution volume of 4.5 L, an operating pressure of 20 Bar, a temperature of 20° C., and a concentrated volume of ½ (under this condition, the conductivity and D-glucosamine content of the corn glycopeptides were reduced by 16.87% and 7.58%, respectively).

### Example 2

Hepatoprotective Activity of Corn Glycopeptides:

50 SD rats were randomly divided into 5 groups with 10 rats in each group, namely a normal control group, an alcohol model group, and 3 corn glycopeptides groups which were a group with 250 mg/kg·BW of corn glycopeptides, a group with 500 mg/kg·BW of corn glycopeptides, and a group with 1 g/kg·BW of corn glycopeptides. After one week of adaptive breeding, modeling was started. After oral gavage of the corn glycopeptides for 30 min, the rats in the corn glycopeptides group and the alcohol model group were subjected to gastric perfusion of 50° Beidacang dabuyou liquor purchased from Beidacang Group Co., Ltd., Hei Longjiang at a dose of 0.8 mL/100 g·BW, the dose was adjusted to 1.0 mL/100 g·BW in the second week, to 1.0 mL/100 g·BW in the third week, and to 1.5 mL/100 g·BW in the fourth week, and the gavage period was 28 d. The rats of the normal control groups were subjected to gastric perfusion of normal saline according to the same dose. 12 h after the last feed intake, the rats were sacrificed, and blood and organs were taken to analyze relevant indices.

After placed at room temperature for 24 h, the blood was centrifuged at 3000 r/min for 10 min, and the serum was collected to determine the activity of alanine aminotransferase (ALT) and the activity of aspartate aminotransferase (AST). The liver was mixed with 0.9% normal saline at a ratio of 1:10 (g/mL), and homogenized in an ice bath to form a 10% liver homogenate, which was used to determine the superoxide dismutase (SOD) activity, the hydrogen peroxide (CAT) activity and the reactive oxygen species (ROS) content. The ALT, AST, SOD, CAT and ROS were each determined by the kit available from Shanghai Jianglai industrial Limited By Share Ltd. The determination method followed the manufacturer's instructions of the kit. The experimental results were as shown in Table 1.

TABLE 1
Effects of Corn Glycopeptide on Serum and Hepatic Indices in Rats

| | | | Corn | Corn | Corn |
|---|---|---|---|---|---|
| Detection | Normal | Alcohol | Glycopeptides of | Glycopeptides of | Glycopeptides of |
| Indicator | Group | Model Group | 250 mg/kg · BW | 500 mg/kg · BW | 1 g/kg · BW |
| Hepatic | 33.07 ± 3.30 | 40.38 ± 4.15 | 34.63 ± 3.35 | 34.83 ± 3.50 | 34.63 ± 2.76 |
| index (mg/g) | | | | | |
| ALT (U/L) | 69.16 ± 14.09 | 103.81 ± 26.38 | 60.39 ± 15.59 | 66.12 ± 12.79 | 54.6 ± 6.59 |
| AST (U/L) | 234.93 ± 51.77 | 355.43 ± 167.64 | 245.52 ± 85.85 | 209.13 ± 127.12 | 194.47 ± 47.0 |
| SOD (U/mL) | 199.00 ± 35.49 | 86.70 ± 19.28 | 136.62 ± 9.09 | I45.88 ± 22.69 | 146.78 ± 16.41 |
| CAT (U/mL) | 74.12 ± 16.32 | 36.36 ± 6.60 | 56.21 ± 9.22 | 59.27 ± 7.44 | 60.99 ± 5.64 |
| ROS (U/mL) | 243.82 ± 41.09 | 359.40 ± 33.62 | 277.54 ± 52.00 | 276.04 ± 47.31 | 261.87 ± 12.18 |

The results showed that: compared with the alcohol model group, when the dose of the corn glycopeptides was 1 g/kg·BW, the corn glycopeptides reduced hepatic index and the levels of ALT, AST and ROS of the rats by 14.15%, 47.40%, 43.88% and 27.14% respectively, while increased the activities of SOD and CAT by 69.29% and 67.74% respectively, indicating that the corn glycopeptides played a protective role on the alcoholic liver injury.

The above description is only preferred embodiments of the present disclosure. It should be pointed out that, for those of ordinary skills in the art, several improvements and modifications can be made without departing from the principle of the present disclosure. These improvements and modifications should also be considered as falling into the claimed scope of the present disclosure.

What is claimed is:

1. A method for preparing corn glycopeptides, comprising the following steps:
(1) hydrolyzing a corn protein suspension, conducting liquid-solid separation, and drying a liquid phase to obtain corn peptide powder;
(2) mixing the corn peptides powder with water to formulate a corn peptides solution with a mass-volume concentration of 2-15%;
(3) adding amino sugar into the corn peptides solution to obtain a reaction base solution; wherein the addition amount of the amino sugar is 2-5 times of the mass of the corn peptides powder;
(4) adding a transglutaminase into the reaction base solution to enable a glycosylation reaction between the corn peptides and the amino sugar, so as to obtain the corn glycopeptides after the reaction is finished; wherein the addition amount of the transglutaminase is 40-80 U of the transglutaminase per 1 g of the corn peptides powder.

2. The preparation method according to claim 1, wherein the hydrolytic enzyme in the step (1) comprises one or more of a Alcalase protease, a Protamex protease and a Flavourzyme protease, the hydrolysis temperature is 55-65° C., the pH value of hydrolysis is maintained at 8.2-8.8, and the hydrolysis time is 1-3 h; the maintaining of the pH value is achieved by adding 0.4-0.6 mol/L of a NaOH solution dropwise; and after the hydrolysis, the method further comprises a primary enzyme inactivation, wherein the temperature of the primary enzyme inactivation is 90-105° C., and the time of the primary enzyme inactivation is 10-20 min.

3. The preparation method according to claim 1, wherein the manner of solid-liquid separation in the step (1) is centrifugation, the rotation speed of the centrifugation is 3000-5000 r/min, and the centrifugation time is 10-20 min.

4. The preparation method according to claim 1, wherein after the glycosylation reaction of the step (4) is finished, the method further comprises the steps of centrifuging the reaction solution, taking the supernatant after the centrifuging, and performing nanofiltration of the supernatant; the nanofiltration can cut off molecules with molecular weights ≤300 Da.

5. The preparation method according to claim 4, wherein for the nanofiltration, the pressure is 15-25 Bar, and the temperature is 18-22° C., and the solution volume after the nanofiltration is ⅓-⅔ of the solution volume before the nanofiltration.

6. The preparation method according to claim 1, wherein the temperature of the glycosylation reaction in the step (4) is 42-46° C., the pH value of the glycosylation reaction is maintained at 7.8-8.2, and the time of the glycosylation reaction is 5-10 h.

7. The preparation method according to claim 2, wherein the temperature of the glycosylation reaction in the step (4) is 42-46° C., the pH value of the glycosylation reaction is maintained at 7.8-8.2, and the time of the glycosylation reaction is 5-10 h.

8. The preparation method according to claim 3, wherein the temperature of the glycosylation reaction in the step (4) is 42-46° C., the pH value of the glycosylation reaction is maintained at 7.8-8.2, and the time of the glycosylation reaction is 5-10 h.

9. The preparation method according to claim 4, wherein the temperature of the glycosylation reaction in the step (4) is 42-46° C., the pH value of the glycosylation reaction is maintained at 7.8-8.2, and the time of the glycosylation reaction is 5-10 h.

10. The preparation method according to claim 5, wherein the temperature of the glycosylation reaction in the step (4) is 42-46° C., the pH value of the glycosylation reaction is maintained at 7.8-8.2, and the time of the glycosylation reaction is 5-10 h.

11. The preparation method according to claim 6, wherein the maintaining of the pH value is achieved by adding 3-5 mol/L of the NaOH solution dropwise.

12. The preparation method according to claim 6, wherein the glycosylation reaction further comprises secondary enzyme inactivation, the temperature of the secondary enzyme inactivation is 80-90° C., and the time of the secondary enzyme inactivation is 3-8 min.

13. A corn glycopeptides-containing product prepared by the preparation method according to claim 1, wherein the mass concentration of a protein in the product is 70%-80%, and the protein comprises corn glycopeptides and corn peptides; and the amount of glucosamine conjugated to corn peptides is 100-150 mg per 1 g of the corn peptides.

14. A corn glycopeptides-containing product prepared by the preparation method according to claim 2, wherein the mass concentration of a protein in the product is 70%-80%, and the protein comprises corn glycopeptides and corn peptides; and the amount of glucosamine conjugated to corn peptides is 100-150 mg per 1 g of the corn peptides.

15. A corn glycopeptides-containing product prepared by the preparation method according to claim 3, wherein the mass concentration of a protein in the product is 70%-80%, and the protein comprises corn glycopeptides and corn peptides; and the amount of glucosamine conjugated to corn peptides is 100-150 mg per 1 g of the corn peptides.

16. A corn glycopeptides-containing product prepared by the preparation method according to claim 4, wherein the mass concentration of a protein in the product is 70%-80%, and the protein comprises corn glycopeptides and corn peptides; and the amount of glucosamine conjugated to corn peptides is 100-150 mg per 1 g of the corn peptides.

17. A corn glycopeptides-containing product prepared by the preparation method according to claim 5, wherein the mass concentration of a protein in the product is 70%-80%, and the protein comprises corn glycopeptides and corn peptides; and the amount of glucosamine conjugated to corn peptides is 100-150 mg per 1 g of the corn peptides.

18. A corn glycopeptides-containing product prepared by the preparation method according to claim 6, wherein the mass concentration of a protein in the product is 70%-80%, and the protein comprises corn glycopeptides and corn peptides; and the amount of glucosamine conjugated to corn peptides is 100-150 mg per 1 g of the corn peptides.

19. Application of the corn glycopeptides prepared by the preparation method according to claim 1 in the preparation of health-care food, drugs or health care products for antagonizing alcoholic liver injury.

20. Application of the corn glycopeptides-containing product according to claim 13 in the preparation of health-care food, drugs or health care products for antagonizing alcoholic liver injury.
